(19) United States
(12) Patent Application Publication
(10) Pub. No.: US 2007/0081986 A1
(43) Pub. Date: Apr. 12, 2007

(54) Beta-glucuronidase with an attached short peptide of acidic amino acids

(76) Inventors: Shunji Tomatsu, Missouri, MO (US); Ken'jchi Miyamoto, Ishikawa (JP); Masamichi Yamada, Hyogo (JP); Yasuhiro Tosaka, Hyogo (JP); Mana Yamada, Hyogo (JP); Jeffrey H. Grubb, Missouri, MO (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/245,424

(22) Filed: Oct. 7, 2005

(51) Int. Cl. A61K 38/47; C12N 9/24; C12N 9/42

(52) U.S. Cl. 424/94.61; 435/200; 435/209

(57) ABSTRACT

Disclosed are a fusion protein comprising enzyme β-glucuronidase and short peptide consisting 4-15 acidic amino acids attached to the enzyme on its N-terminal side, pharmaceutical composition containing the fusion protein, and a method for treatment of type VII mucopolysaccharidosis using the fusion protein. Compared with the native enzyme, the fusion protein exhibits higher stability in the blood.

### TECHNICAL FIELD

The present invention relates to endowing β-glucuronidase protein with increased in vivo stability. More specifically, the present invention relates to endowing β-glucuronidase protein with improved stability in the blood by attaching a short peptide consisting of acidic amino acids to the N-terminus of the protein.

### BACKGROUND ART

It has been reported that acidic peptide chains consisting of aspartic acid and/or glutamic acid molecules have high bonding affinities for hydroxyapatite, one of the component materials of the bone (1,2). Making use of this property, techniques have been reported by which those acidic peptide chains are attached to steroid hormones (sex hormones or protein anabolic hormones, etc.), which are used for bone diseases such as osteoporosis, for endowing those steroid hormones with bone-tissue targeting ability (Japanese Patent Application Publication No. 2000-327583)(3). Further techniques have been reported by which peptide chains made of carboxylated amino acid derivatives having three or more carboxyl groups per molecule were attached to, and used as bone-targeting, drug-transporting carriers for, steroid hormones, methotrexate, anti-cancer antibiotics, alkylating agents or cell growth factors (Japanese Patent Application Publication No. 2002-3407)(4).

Meanwhile there is a problem that pharmaceutical preparations of physiologically active proteins like enzymes and peptide hormones are generally made unstable when they are administered to the body, and thus undergo relatively rapid inactivation by, e.g., enzymatic degradation. For pharmaceutical preparations of a physiologically active protein, a method for increasing the stability of the physiologically active protein in the body is known which is based on coupling the proteins to polyethylene glycol (Japanese Patent No. 2852127)(5).

Sly's syndrome is an autosomal recessive, genetic disease caused by an anomaly in the gene for a lysosomal enzyme, β-glucuronidase (hereinafter referred to as GUS) (6), and is classified as type VII mucopolysaccharidosis (hereinafter referred to as MPS VII). In lysosomes, GUS acts as an exoglycosidase to remove glucuronic acid residues from the non-reducing termini of GAGs (glycosaminoglycans), such as dermatan sulfate (DS), heparan sulfate (HS), and chondroitin sulfate (CS). In the absence of GUS, GAGs is only partially degraded and accumulates in lysosomes of a variety of tissues. Progressive accumulation of undegraded GAGs in lysosomes affects the spleen, liver, kidney, cornea, brain, heart valves, and the skeletal system, leading to facial dysmorphism, growth retardation, systemic bone dysplasia, deafness, mental retardation, and shortened lifespan.

No effective remedy is currently available for MPS VII. The enzyme substitution therapy has been considered to be the potential remedy for MPS VII. Considering its rapid inactivation in the body, however, native GUS is not expected to give any satisfactory effect.

### DISCLOSURE OF INVENTION

Against the above-mentioned background, an objective of the present invention is to increase in vivo stability of physiologically active GUS administered to a patient with MPS VII. With acidic short peptide attached to the N-terminus of GUS, the inventors unexpectedly found that it improves in great deal the in vivo stability of GUS. The present invention was completed upon the finding.

Thus the present invention provides:

1. A fusion protein comprising
a physiologically active GUS and
a short peptide which consists of 4-15 acidic amino acids and is attached to the physiologically active human β-glucuronidase on the N-terminal side thereof.

2. The fusion protein according to 1 above, wherein the short peptide is attached to the N-terminus of the physiologically active human β-glucuronidase via a linker peptide.

3. A method for increasing the stability of physiologically active human β-glucuronidase administered in the blood, wherein the method comprises converting the physiologically active human β-glucuronidase into a fusion protein comprising
a physiologically active human β-glucuronidase and
a short peptide which consists of 4-15 acidic amino acids and is attached to the physiologically active human β-glucuronidase on the N-terminal side thereof.

4. The method according to 3 above, wherein the short peptide is attached to the N-terminus of physiologically active human β-glucuronidase via a linker peptide.

5. A pharmaceutical composition comprising a fusion protein comprising
a physiologically active human β-glucuronidase and
a short peptide which consists of 4-15 acidic amino acids and is attached to the physiologically active human β-glucuronidase on the N-terminal side thereof.

6. The pharmaceutical composition according to 5 above, wherein the short peptide is attached to N-terminus of the physiologically active human β-glucuronidase via a linker peptide.

7. A method for treatment of type VII mucopolysaccharidosis in a human patient comprising administering to the human patient a therapeutically effective amount of the fusion protein according to claim 1 or 2.

Comparing with native physiologically active GUS, the present invention described above provides physiologically active fusion proteins with increased stability in the blood when administered to a patient with MPS VII. The present invention further provides a pharmaceutical composition useful for the treatment of MPS VII in human patients, as well as a method for the treatment of MPS VII.

### BRIEF DESCRIPTION OF DRAWINGS

FIG. 1 is a schematic diagram illustrating pCXN vector and the cloning site in the vector for the cDNA encoding native GUS or the GUS fusion protein.

FIG. 2 illustrates the steps for the construction of an expression vector for the production of the GUS and GUS fusion protein.

FIG. 3 is a graph showing the time profiles of the blood activity levels of native GUS and GUS fusion protein after they are intravascularly administered in an equivalent amount.

### BEST MODE FOR CARRYING OUT THE INVENTION

The term “Acidic amino acid” referred to the present invention means glutamic acid or aspartic acid. As the employment of these acidic amino acids in the present invention is for the purpose of constructing an acidic short peptide, they may be used in any arbitrary combination including a simple use of one or the other of them alone for construction of such a short peptide. The number of the acidic amino acids forming a short peptide is preferably 4-15, more preferably 4-12, and still more preferably 4-8.

A short peptide consisting of acidic amino acids may be directly attached to the N-terminus of physiologically active human GUS via a peptide bond or like, or, instead, it may be attached via a linker peptide.

In the present invention “a linker peptide” is not an indispensable component, for it is usable only for convenience in attaching a short peptide consisting of acidic amino acids to N-terminus of physiologically active GUS. Where it is used, a linker peptide may be a short peptide consisting e.g., preferably of 15 or less, more preferably of 10 or less, and still more preferably of 6 or less amino acids. Such a linker that consists of a single amino acid molecule and linking between the acidic short peptide and physiologically active GUS via peptide bonds is also included in the definition of a linker peptide. A linker peptide may be made of any one or more amino acids desired.

In the present invention, though there is no specific limitation as to the method for attaching an acidic short peptide to physiologically active GUS, it is of advantage, e.g., to form and use a transformant cell expressing the fusion protein consisting of the short peptide and physiologically active GUS.

A fusion protein of the present invention may include a non-acidic amino acid or a sequence of several (e.g., 3) non-acidic amino acids at N-terminus of the short peptide consisting of acidic amino acids.

A fusion protein of the present invention may be formulated into a pharmaceutical composition containing the fusion protein dissolved or dispersed in a pharmaceutically acceptable carrier well known to those skilled in the art, for parenteral administration by e.g., intravenous, subcutaneous, or intramuscular injection or by intravenous drip infusion.

For pharmaceutical compositions for parenteral administration, any conventional additives may be used such as excipients, binders, disintegrants, dispersing agent, lubricants, diluents, absorption enhancers, buffering agents, surfactants, solubilizing agents, preservatives, emulsifiers, isotonizers, stabilizers, solubilizers for injection, pH adjusting agents, etc.

A fusion protein of the present invention may be used advantageously in place of the conventional native enzyme protein in a substitution therapy for the treatment of MPS VII. In the treatment, the fusion protein may be administered intravenously, subcutaneously, or intramuscularly. Doses and frequencies of administration are to be determined by the physician in charge in accordance with the condition of his or her patient.

### EXAMPLES

[Method for Construction of Expression Vectors]

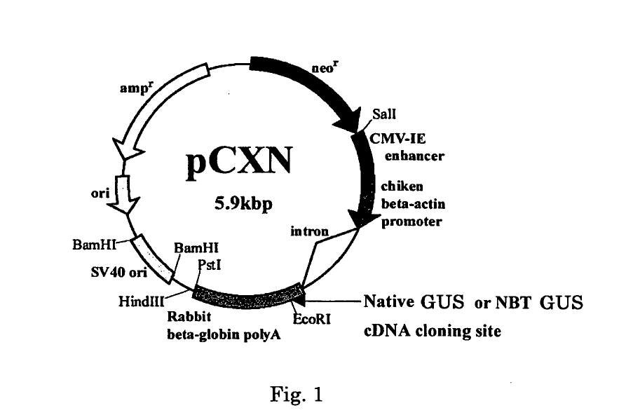

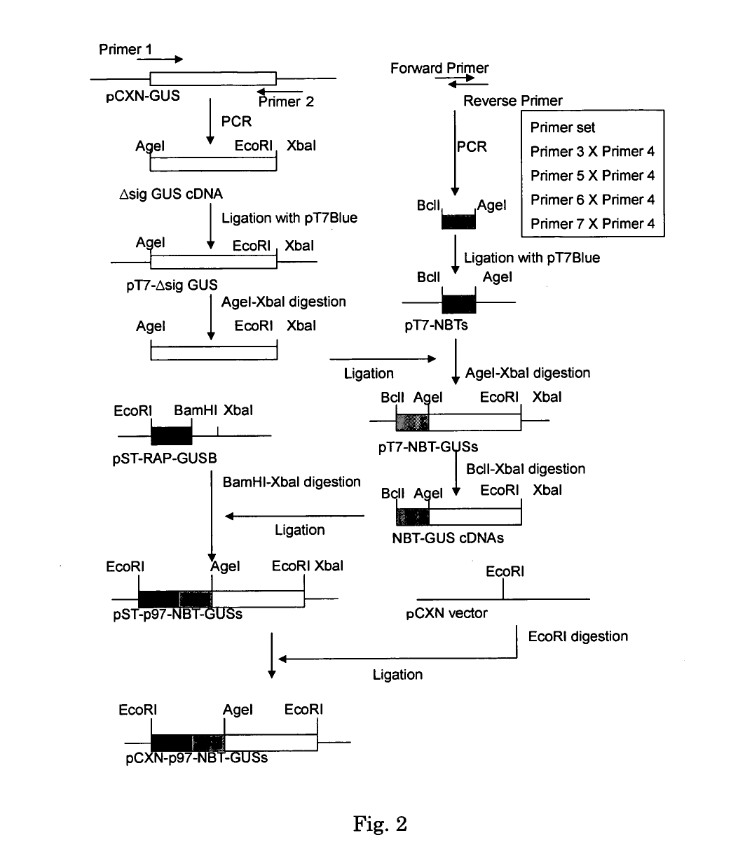

Vector pCXN had been constructed in accordance with a literature (7) and was offered to us by Prof. Miyazaki at Osaka University. An expression vector for native human GUS, pCXN-GUS, was constructed by using human GUS cDNA that had been reported by Oshima et al. (8)(Accession No. of GenBank for the Amino acid and cDNA sequence of Human GUS is BC014142.). An expression vector for human GUS to the N-terminus of which is attached (via a linker peptide) a short peptide (N-terminal bone tag: NBT) consisting of acidic amino acids (NBT-GUS), was constructed starting with pCXN-GUS in the following manner. FIGS. 1 and 2 schematically illustrate the process for construction.

Using pCXN-GUS as a template, PCR was carried out using LA-Taq (Takara) to amplify Δsig GUS cDNA (the sequence, nt 67-1956, left behind after removal of the sequence of nt 1-66 corresponding to a secretion signal, from the ORF region of the sequence set forth as SEQ ID NO:1) (for human GUS without signal sequence, see SEQ ID NO:2), to the 5′-terminus of which is attached an AgeI cleavage sequence. The PCR was carried out according to the instruction for use of LA-Taq, i.e., through the cycles consisting of 30 seconds at 94° C., (30 seconds at 94° C., 30 seconds at 60° C., and 2 minutes at 72° C.)×25, and then 3 minutes at 72° C., with primer 1 (SEQ ID NO:3), and primer 2 (SEQ ID NO:4). The cDNA thus amplified was inserted into pT7Blue vector (Novagen) to construct pT7-Δsig GUS.

The N-terminal bone tag (NBT) cDNA to be attached to the 5′-terminus then was constructed by PCR using LA-Taq (Takara). Briefly, primer 3 (SEQ ID NO:5) and primer 4 (SEQ ID NO:6) were used for the construction of NBT-E6 cDNA, primer 5 (SEQ ID NO:7) and primer 4 (SEQ ID NO:6) for the construction of NBT-E8 cDNA, primer 6 (SEQ ID NO:8) and primer 4 (SEQ ID NO:6) for the construction of NBT-D6 CDNA, and primer 7 (SEQ ID NO:9) and primer 4 (SEQ ID NO:6) for the construction of NBT-D8 cDNA. In the names of the NBT cDNAs, “E6” or “E8” indicate that the NBT is made up of 6 or 8 serially connected glutamic acid residues, respectively. Likewise, “D6” or “D8” indicates that the NBT is made up of 6 or 8 connected aspartic acid residues, respectively.

Employing each pair of the above primers, which contained a portion complementary to each other, PCR was carried out through the cycles consisting of 30 seconds at 94° C., (30 seconds at 94° C., 30 seconds at 60° C., 30 seconds at 72°)×20 minutes, and then one minute at 72° C. The thus amplified DNA fragments were inserted into pT7Blue vector (Novagen) to construct pT7-NBTs.

A human GUS cDNA recovered as a fragment of pT7-Δsig GUS cleaved with AgeI and XbaI was inserted into the AgeI-XbaI site of pT7-NBTs to construct pT7-NBT-GUSs. Then each of pT7-NBT-GUSs was cleaved with BclI, blunt-ended with T4 DNA polymerase, and cleaved with XbaI to recover NBT GUS cDNAs.

pST-RAP-GUSB (a vector comprising the p97 signal sequence, provided by Tomatsu at Saint Louis University) was cleaved with BamHI and XbaI, into which then was inserted the NBT-GUS cDNAs recovered above to construct pST-p97- NBT-GUSs.

pST-p97-NBT-GUSs were cleaved with EcoRI to recover respective p97-NBT-GUS cDNAs, each of which then was inserted into the EcoRI site of pCXN to construct a NBT-GUS expression vector, pCXN-p97-NBT-GUS. The DNA sequence of the expression vectors' region corresponding to the p97-NBT-D6-GUS, p97-NBT-D8-GUS, p97-NBT-E6-GUS and p97-NBT-E8-GUS cDNAs are shown in the Sequence Listing (SEQ ID NO:10, SEQ ID NO:12, SEQ ID NO:14, SEQ ID NO:16,) along with their corresponding amino acid sequences (SEQ ID NO:11, SEQ ID NO:13, SEQ ID NO:15, SEQ ID NO:17), respectively.

SEQ ID NO:10 shows part of the sequence containing the NBT-E6-GUS cDNA of pCXN-p97-NBT-E6-GUS. Its nt 1-57 encode the p97 signal sequence, nt 61-78 a poly Glu, nt 79-96 a linker sequence, and nt 97-1983 GUS without the signal sequence.

SEQ ID NO:11 shows the NBT-E6-GUS amino acid sequence with the p97 signal sequence. Aa 1-19: p97 signal sequence, aa2l-26: poly Glu, aa 27-32: linker sequence, aa 33-661: GUS without signal sequence.

SEQ ID NO:12 shows part of the sequence containing the NBT-E8-GUS cDNA of pCXN-p97-NBT-E8-GUS. Its nt 1-57 encode the p97 signal sequence, nt 61-84 a poly Glu, nt 85-102 a linker sequence, and nt 103-1989 GUS without the signal sequence.

SEQ ID NO:13 shows the NBT-E8-GUS amino acid sequence with attached p97 signal sequence. Aa 1-19: p97 signal sequence, aa 21-28: poly Glu, aa 29-34: linker sequence, aa 35-663: GUS without signal sequence.

SEQ ID NO:14 shows part of the sequence containing the NBT-D6-GUS cDNA of pCXN-p97-NBT-D6-GUS. Its nt 1-57 encode the p97 signal sequence, nt 61-78 a poly Asp, nt 79-96 a linker sequence, and nt 97-1983 GUS without the signal sequence.

SEQ ID NO:15 shows the NBT-D6-GUS amino acid sequence with attached p97 signal sequence. Aa 1-19: p97 signal sequence, aa2l-26: poly Asp, aa 27-32: linker sequence, aa 33-661: GUS without signal sequence.

SEQ ID NO:16 shows part of the sequence containing the NBT-D8-GUS cDNA of pCXN-p97-NBT-D8-GUS. Its nt 1-57 encode the p97 signal sequence, nt 61-84 a poly Asp, nt 85-102 a linker sequence, and nt 103-1989 GUS without the signal sequence.

SEQ ID NO:17 shows the NBT-D8-GUS amino acid sequence with attached p97 signal sequence. Aa 1-19: p97 signal sequence, aa 21-28: poly Asp, aa 29-34: linker sequence, aa 35-663: GUS without signal sequence.

The proteins set forth as SEQ ID NO:11, SEQ ID NO:13, SEQ ID NO:15 and SEQ ID NO:17 contain the p97 secretion signal sequence. The signal sequence is removed during secretion process from the cell and the fusion proteins are thus recovered as NBT-GUS in the medium.

p97 is a cell-surface glycoprotein occurring in most human melanomas and its signal sequence consists of 19 amino acids(9). The aforementioned pCXN-p97-NBT-GUSs containing the cDNA encoding this signal sequence may also be constructed by the following method. Briefly, a cDNA containing the p97 signal sequence is synthesized through the process of primers annealing and PCR amplification. LA-Taq is used as an enzyme for PCR. As primers having mutually complementary portions, primer 8 (SEQ ID NO:18) and primer 9 (SEQ ID NO:19) are used. PCR is performed through the cycles of 30 seconds at 94° C., (30 seconds at 94° C., 30 seconds at 60° C., 30 seconds at 72° C.)×20, and one minute at 72° C. The amplified cDNA containing the p97 signal sequence is cleaved with BamHI and EcoRI. Into the pCXN vector, after cleaved with EcoRI, are simultaneously incorporated the aforementioned NBT-GUSs cDNA recovered after the enzyme treatment and cDNA for the p97 signal sequence, giving pCXN-p97-NBT-GUSs.

SEQ ID No:18 is a forward primer, in which nt 1-5 comprise a random synthetic sequence, and nt 6-52 comprise part of the sequence encoding the p97 signal.

SEQ ID No:19 is a reverse primer, in which nt 1-6 comprise a random synthetic sequence, and nt 7-52 comprise part of the sequence encoding the p97 signal.

[Establishment of Expression Cells]

Nunclon delta-MultiDish 6 Well was inoculated with CHO-K1 cells. After an overnight culture in DMEM/F12/FBS medium [DMEM/F12 medium(Gibco) supplemented with 10% fetal bovine serum (Thermo Trace)], each of the expression vector constructed above was introduced into the cells using Lipofectamine 2000 reagent. For experimental procedures, the instruction manual attached to the Lipofectamine 2000 reagent was followed. After a two-day incubation at 37° C. in 5% CO₂, the cells were added to 75-cm² tissue culture flasks (Iwaki) and incubated until colonies of resistant cells were formed with Geneticin (Gibco) added to the DMEM/F12/FBS medium at the final concentration of 1 mg/mL. After formation of colonies was confirmed under a microscope, cells which exhibited stable expression were cloned by the limiting dilution-culture method. Screening for expression cells were performed by GUS-specific enzyme activity assay of the culture supernatants. Cell lines thus established were subcultured in DMEM/F12/FBS medium supplemented with 0.2 mg/mL Geneticin.

[Method for Measurement of GUS-Specific Enzyme Activity]

After intravenous administration of native- or NBT-GUS to mice, GUS activity in the blood was determined as follows. Briefly, 12.5 uL of plasma sample from the mice was added to 50 uL of a solution of 10 mM 4-methylumbelliferyl-β-D-glucuronide (Sigma Chemical Co., St. Louis, Mo., cat # M9130) which had been prepared using determination buffer (0.1M sodium acetate buffer pH 4.8), and reaction was allowed for 1 hr at 37° C. Then, 950 uL of stop buffer (1 M Glycine-HCl, pH 10.5) was added and mixed to stop the enzyme reaction. Samples of the reaction mixture were transferred to a fluorometer for measurement with excitation 366 nm/emission 450 nm.

[Expression and Purification of Native GUS and GUS Fusion Protein]

Native GUS and GUS fusion proteins were produced in overexpressing CHO cells, which were grown to confluency and fed with low-serum medium (Waymouth's MB 752/1 medium, supplemented with 2% FBS/1.2 mM glutamine/1 mM pyruvate) (Gibco) for purification every 24 hr. The media of the culture were pooled, centrifuged at 5,000×g for 20 min at 4° C., and frozen at −20° C. Purification was performed using affinity chromatography (10). Briefly, the conditioned medium from cells overexpressing the Native GUS or a GUS fusion protein was filtered, and NaCl was added to the medium at the final concentration of 0.5 M. The medium was applied to a 5 ml column of Affi-Gel 10 (BioRad) which carried an anti-human GUS monoclonal antibody and had been pre-equilibrated with wash buffer. The column was washed at 36 mL/hour with 20-column volumes of wash buffer. The column was eluted at 36 mL/hour with 50 ml of 10 mM sodium phosphate (pH 5.0) containing 3.5 M MgCl₂. Fractions were collected and subjected to GUS activity assay. Fractions containing the enzyme activity were pooled for each of the Native or fusion proteins, diluted with an equal volume of P6 buffer (25 mM Tris, pH 7.5/1 mM β-glycerol phosphate/0.15 mM NaCl/0.025% sodium azide), and desalted over a BioGel P6 column (BioRad) pre-equilibrated with P6 buffer. Fractions containing GUS activity were pooled, and the finally purified active protein was stored at −80° C.

[Stability in the Blood]

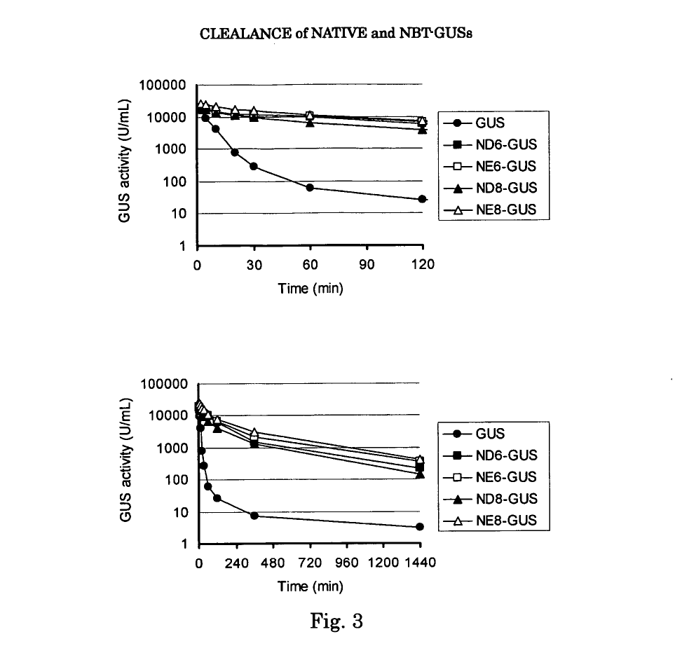

Per 1 g of body weight, 1,000 U of native GUS or one of the NBT-GUSs, both purified, were administered to male, 4-month old C57BL mice (3 animals/group) in the tail vein. Samples of venous blood were collected at 2 min, 5 min, 10 min, 20 min, 30 min, 1 hr, 2 hr, 6 hr, 24 hr after the administration, and GUS activity in the serum was measured. The results are shown in FIG. 3. Comparison between the NBT-GUSs-administered groups and the native GUS-administered group reveales that, at 2 min after the administration, the enzyme activity in the blood was 2-fold higher in the NBT-GUSs-administered groups as compared with the native GUS-administered group. And, while the enzyme activity in the blood at 30 min after the administration was almost disappeared in the native GUS-administered group, the NBT-GUSs-administered groups retained activity levels, which were even higher than the activity level found at 2 min in the native GUS-administered group. Afterwards, the NBT-GUSs-administered groups continued to show remarkably slower reduction in the enzyme activity levels in the blood as compared with those found in the native GUS-administered group. Even 24 hr (1440 min) after the administration, the residual enzyme activity was detectable in the NBT-GUSs-administered group. A half-life time of the enzyme activity in blood in the native GUS-administered group was 4.9 min, while a half-life time in blood in the NBT-GUS-administered group was prolonged 5-6 times. The results demonstrate that the stability of GUS in the body is remarkably increased by attaching a short peptide of acidic amino acids to the N-terminus of native GUS.

### INDUSTRIAL APPLICABILITY

The present invention enables production of physiologically active proteins having the enzyme activity of GUS and having increased stability in the body. The present invention also provides a method and a pharmaceutical composition for treatment of MPS VII.

[References]

(1) Bernardi G, Chromatography of protein on hydroxyapatite. Method Enzymol. 27: 471-9 (1973)
(2) Fujisawa R, Wada Y, Nodasaka Y, Kuboki Y, Acidic amino acid-rich sequences as binding sites of osteonectin to hydroxyapatite crystals. Biocem Biopys Acta 41292:53-60 (1996)
(3) Japanese Patent Application Publication No. 2000-327583
(4) Japanese Patent Application Publication No. 2002-3407
(5) Japanese Patent No. 2852127
(6) Sly W S, Quinton B A, McAlister W H, and Rimoin D L, Beta-glucuronidase deficiency: report of clinical, radiologic, and biochemical features of a new mucopolysaccharidosis. J Pediatr. 82:249-257 (1973)
(7) Niwa H, Yamamura K, Miyazaki J, Efficient selection for high-expression transfentacts with a novel eukaryotic vector. Gene 108: 193-200 (1991)
(8) Oshima A, Kyle J W, Miller R D, Hoffmann J W, Powell P P, Grubb JH, Sly W S, Tropak M, Guise K S, Gravel. Cloning, sequencing, and expression of cDNA for human beta-glucuronidase. Proc Natl Acad Sci U S A. 84: 685-689
(9) Rose, T. M., et al., Primary structure of human melanoma-associated antigen p97 (melanotransferrin) deduced from the mRNA sequence, Proc. Natl. Acad. Sci. USA, 83:1261-1265 (1986)
(10) LeBowitz J H, Grubb J H, Maga J A, Schmiel D H, Voglar C, Sly W S, Glycosylation-independent targeting enhances enzyme delivery to lysosomes and decreases storage in mucopolysaccharidosis type VII mice. Proc Natl Acad Sci USA. 101: 3083-3086. (2004)

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosure[s] of all applications, patents and publications, cited herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

We claim:

1. A fusion protein comprising
a physiologically active human 6-glucuronidase and
a short peptide which consists of 4-12 acidic amino acids and is attached to the physiologically active human β-glucuronidase on the N-terminal side thereof.

2. The fusion protein according to claim 1, wherein the short peptide is attached to the N-terminus of the physiologically active human β-glucuronidase via a linker peptide.

3. A method for increasing the stability of physiologically active human β-glucuronidase administered in the blood, wherein the method comprises converting the physiologically active human β-glucuronidase into a fusion protein comprising
a physiologically active human β-glucuronidase and
a short peptide which consists of 4-12 acidic amino acids and is attached to the physiologically active human β-glucuronidase on the N-terminal side thereof.

4. The method according to claim 3, wherein the short peptide is attached to the N-terminus of physiologically active human β-glucuronidase via a linker peptide.

5. A pharmaceutical composition comprising a fusion protein of claim 1 and a pharmaceutically acceptable carrier or excipient.

6. The pharmaceutical composition according to claim 5, wherein the short peptide is attached to N-terminus of the physiologically active human β-glucuronidase via a linker peptide.

7. A method for treatment of type VII mucopolysaccharidosis in a human patient comprising administering to the human patient a therapeutically effective amount of the fusion protein according to claim 1.

8. A method for treatment of type VII mucopolysaccharidosis in a human patient comprising administering to the human patient a therapeutically effective amount of the pharmaceutical composition according to claim 5.

9. A fusion protein comprising
a physiologically active human 6-glucuronidase and
a short peptide which consists of 4-12 acidic amino acids and is attached to the physiologically active human β-glucuronidase on the N-terminal side thereof wherein said fusion protein has β-glucuronidase activity.

10. The fusion protein according to claim 9, wherein the short peptide is attached to the N-terminus of the physiologically active human β-glucuronidase via a linker peptide.

11. A pharmaceutical composition comprising a fusion protein of claim 9 and a pharmaceutically acceptable carrier or excipient.

12. A method for treatment of type VII mucopolysaccharidosis in a human patient comprising administering to the human patient a therapeutically effective amount of the fusion protein according to claim 9.

13. A method for treatment of type VII mucopolysaccharidosis in a human patient comprising administering to the human patient a therapeutically effective amount of the pharmaceutical composition according to claim 11.


SEQUENCE LISTING (19 sequences)

SEQ ID NO: 1 - DNA, 1956 bp, Homo sapiens
  sig_peptide (1)..(66)
  CDS (67)..(1953): Coding region for mature GUS
atggcccgggggtcggcggttgcctgggcggcgctcgggccgttgttgtggggctgcgcgctggggctgcagggcgggatgctgtacccccaggagagcccgtcgcgggagtgcaaggagctggacggcctctggagcttccgcgccgacttctctgacaaccgacgccggggcttcgaggagcagtggtaccggcggccgctgtgggagtcaggccccaccgtggacatgccagttccctccagcttcaatgacatcagccaggactggcgtctgcggcattttgtcggctgggtgtggtacgaacgggaggtgatcctgccggagcgatggacccaggacctgcgcacaagagtggtgctgaggattggcagtgcccattcctatgccatcgtgtgggtgaatggggtcgacacgctagagcatgaggggggctacctccccttcgaggccgacatcagcaacctggtccaggtggggcccctgccctcccggctccgaatcactatcgccatcaacaacacactcacccccaccaccctgccaccagggaccatccaatacctgactgacacctccaagtatcccaagggttactttgtccagaacacatattttgactttttcaactacgctggactgcagcggtctgtacttctgtacacgacacccaccacctacatcgatgacatcaccgtcaccaccagcgtggagcaagacagtgggctggtgaattaccagatctctgtcaagggcagtaacctgttcaagttggaagtgcgtcttttggatgcagaaaacaaagtcgtggcgaatgggactgggacccagggccaacttaaggtgccaggtgtcagcctctggtggccgtacctgatgcacgaacgccctgcctatctgtattcattggaggtgcagctgactgcacagacgtcactggggcctgtgtctgacttctacacactccctgtggggatccgcactgtggctgtcaccaagagccagttcctcatcaatgggaaacctttctatttccacggtgtcaacaagcatgaggatgcggacatccgagggaagggcttcgactggccgctgctggtgaaggacttcaacctgcttcgctggcttggtgccaacgctttccgtaccagccactacccctatgcagaggaagtgatgcagatgtgtgaccgctatgggattgtggtcatcgatgagtgtcccggcgtgggcctggcgctgccgcagttcttcaacaacgtttctctgcatcaccacatgcaggtgatggaagaagtggtgcgtagggacaagaaccaccccgcggtcgtgatgtggtctgtggccaacgagcctgcgtcccacctagaatctgctggctactacttgaagatggtgatcgctcacaccaaatccttggacccctcccggcctgtgacctttgtgagcaactctaactatgcagcagacaagggggctccgtatgtggatgtgatctgtttgaacagctactactcttggtatcacgactacgggcacctggagttgattcagctgcagctggccacccagtttgagaactggtataagaagtatcagaagcccattattcagagcgagtatggagcagaaacgattgcagggtttcaccaggatccacctctgatgttcactgaagagtaccagaaaagtctgctagagcagtaccatctgggtctggatcaaaaacgcagaaaatacgtggttggagagctcatttggaattttgccgatttcatgactgaacagtcaccgacgagagtgctggggaataaaaaggggatcttcactcggcagagacaaccaaaaagtgcagcgttccttttgcgagagagatactggaagattgccaatgaaaccaggtatccccactcagtagccaagtcacaatgtttggaaaacagcctgtttacttga

SEQ ID NO: 2 - protein, 629 aa, Homo sapiens
LQGGMLYPQESPSRECKELDGLWSFRADFSDNRRRGFEEQWYRRPLWESGPTVDMPVPSSFNDISQDWRLRHFVGWVWYEREVILPERWTQDLRTRVVLRIGSAHSYAIVWVNGVDTLEHEGGYLPFEADISNLVQVGPLPSRLRITIAINNTLTPTTLPPGTIQYLTDTSKYPKGYFVQNTYFDFFNYAGLQRSVLLYTTPTTYIDDITVTTSVEQDSGLVNYQISVKGSNLFKLEVRLLDAENKVVANGTGTQGQLKVPGVSLWWPYLMHERPAYLYSLEVQLTAQTSLGPVSDFYTLPVGIRTVAVTKSQFLINGKPFYFHGVNKHEDADIRGKGFDWPLLVKDFNLLRWLGANAFRTSHYPYAEEVMQMCDRYGIVVIDECPGVGLALPQFFNNVSLHHHMQVMEEVVRRDKNHPAVVMWSVANEPASHLESAGYYLKMVIAHTKSLDPSRPVTFVSNSNYAADKGAPYVDVICLNSYYSWYHDYGHLELIQLQLATQFENWYKKYQKPIIQSEYGAETIAGFHQDPPLMFTEEYQKSLLEQYHLGLDQKRRKYVVGELIWNFADFMTEQSPTRVLGNKKGIFTRQRQPKSAAFLLRERYWKIANETRYPHSVAKSQCLENSLFT

SEQ ID NO: 3 - DNA, 35 bp, Homo sapiens
  misc_feature: Primer sequence
gaggcaaccggtctgcagggcgggatgctgtaccc

SEQ ID NO: 4 - DNA, 44 bp, Homo sapiens
  misc_feature: Primer sequence
tctagagaattcctcgagtcaagtaaacaggctgttttccaaac

SEQ ID NO: 5 - DNA, 52 bp, Homo sapiens
  misc_feature: Primer sequence
attaggtacctgatcagaagaggaggaagaagaggccgaggcagaaaccggt

SEQ ID NO: 6 - DNA, 25 bp, Homo sapiens
  misc_feature: Primer sequence
tgcggggcaccggtttctgcctcgg

SEQ ID NO: 7 - DNA, 58 bp, Homo sapiens
  misc_feature: Primer sequence
attaggtacctgatcagaagaggaagaggaggaagaagaggccgaggcagaaaccggt

SEQ ID NO: 8 - DNA, 51 bp, Homo sapiens
  misc_feature: Primer sequence
attaggtacctgatcagatgatgatgatgatgatgccgaggcagaaaccgg

SEQ ID NO: 9 - DNA, 57 bp, Homo sapiens
  misc_feature: Primer sequence
attaggtacctgatcagatgatgatgatgatgatgatgatgccgaggcagaaaccgg

SEQ ID NO: 10 - DNA, 1983 bp, Homo sapiens
  CDS (1)..(1983)
atgcggggtccgagcggggctctgtggctgctcctggctctgcgcaccgtgctcggatcagaagaggaggaagaagaggccgaggcagaaaccggtctgcagggcgggatgctgtacccccaggagagcccgtcgcgggagtgcaaggagctggacggcctctggagcttccgcgccgacttctctgacaaccgacgccggggcttcgaggagcagtggtaccggcggccgctgtgggagtcaggccccaccgtggacatgccagttccctccagcttcaatgacatcagccaggactggcgtctgcggcattttgtcggctgggtgtggtacgaacgggaggtgatcctgccggagcgatggacccaggacctgcgcacaagagtggtgctgaggattggcagtgcccattcctatgccatcgtgtgggtgaatggggtcgacacgctagagcatgaggggggctacctccccttcgaggccgacatcagcaacctggtccaggtggggcccctgccctcccggctccgaatcactatcgccatcaacaacacactcacccccaccaccctgccaccagggaccatccaatacctgactgacacctccaagtatcccaagggttactttgtccagaacacatattttgactttttcaactacgctggactgcagcggtctgtacttctgtacacgacacccaccacctacatcgatgacatcaccgtcaccaccagcgtggagcaagacagtgggctggtgaattaccagatctctgtcaagggcagtaacctgttcaagttggaagtgcgtcttttggatgcagaaaacaaagtcgtggcgaatgggactgggacccagggccaacttaaggtgccaggtgtcagcctctggtggccgtacctgatgcacgaacgccctgcctatctgtattcattggaggtgcagctgactgcacagacgtcactggggcctgtgtctgacttctacacactccctgtggggatccgcactgtggctgtcaccaagagccagttcctcatcaatgggaaacctttctatttccacggtgtcaacaagcatgaggatgcggacatccgagggaagggcttcgactggccgctgctggtgaaggacttcaacctgcttcgctggcttggtgccaacgctttccgtaccagccactacccctatgcagaggaagtgatgcagatgtgtgaccgctatgggattgtggtcatcgatgagtgtcccggcgtgggcctggcgctgccgcagttcttcaacaacgtttctctgcatcaccacatgcaggtgatggaagaagtggtgcgtagggacaagaaccaccccgcggtcgtgatgtggtctgtggccaacgagcctgcgtcccacctagaatctgctggctactacttgaagatggtgatcgctcacaccaaatccttggacccctcccggcctgtgacctttgtgagcaactctaactatgcagcagacaagggggctccgtatgtggatgtgatctgtttgaacagctactactcttggtatcacgactacgggcacctggagttgattcagctgcagctggccacccagtttgagaactggtataagaagtatcagaagcccattattcagagcgagtatggagcagaaacgattgcagggtttcaccaggatccacctctgatgttcactgaagagtaccagaaaagtctgctagagcagtaccatctgggtctggatcaaaaacgcagaaaatatgtggttggagagctcatttggaattttgccgatttcatgactgaacagtcaccgacgagagtgctggggaataaaaaggggatcttcactcggcagagacaaccaaaaagtgcagcgttccttttgcgagagagatactggaagattgccaatgaaaccaggtatccccactcagtagccaagtcacaatgtttggaaaacagcccgtttact

SEQ ID NO: 11 - protein, 661 aa, Homo sapiens
MRGPSGALWLLLALRTVLGSEEEEEEAEAETGLQGGMLYPQESPSRECKELDGLWSFRADFSDNRRRGFEEQWYRRPLWESGPTVDMPVPSSFNDISQDWRLRHFVGWVWYEREVILPERWTQDLRTRVVLRIGSAHSYAIVWVNGVDTLEHEGGYLPFEADISNLVQVGPLPSRLRITIAINNTLTPTTLPPGTIQYLTDTSKYPKGYFVQNTYFDFFNYAGLQRSVLLYTTPTTYIDDITVTTSVEQDSGLVNYQISVKGSNLFKLEVRLLDAENKVVANGTGTQGQLKVPGVSLWWPYLMHERPAYLYSLEVQLTAQTSLGPVSDFYTLPVGIRTVAVTKSQFLINGKPFYFHGVNKHEDADIRGKGFDWPLLVKDFNLLRWLGANAFRTSHYPYAEEVMQMCDRYGIVVIDECPGVGLALPQFFNNVSLHHHMQVMEEVVRRDKNHPAVVMWSVANEPASHLESAGYYLKMVIAHTKSLDPSRPVTFVSNSNYAADKGAPYVDVICLNSYYSWYHDYGHLELIQLQLATQFENWYKKYQKPIIQSEYGAETIAGFHQDPPLMFTEEYQKSLLEQYHLGLDQKRRKYVVGELIWNFADFMTEQSPTRVLGNKKGIFTRQRQPKSAAFLLRERYWKIANETRYPHSVAKSQCLENSPFT

SEQ ID NO: 12 - DNA, 1989 bp, Homo sapiens
  CDS (1)..(1989)
atgcggggtccgagcggggctctgtggctgctcctggctctgcgcaccgtgctcggatcagaagaggaagaggaggaagaagaggccgaggcagaaaccggtctgcagggcgggatgctgtacccccaggagagcccgtcgcgggagtgcaaggagctggacggcctctggagcttccgcgccgacttctctgacaaccgacgccggggcttcgaggagcagtggtaccggcggccgctgtgggagtcaggccccaccgtggacatgccagttccctccagcttcaatgacatcagccaggactggcgtctgcggcattttgtcggctgggtgtggtacgaacgggaggtgatcctgccggagcgatggacccaggacctgcgcacaagagtggtgctgaggattggcagtgcccattcctatgccatcgtgtgggtgaatggggtcgacacgctagagcatgaggggggctacctccccttcgaggccgacatcagcaacctggtccaggtggggcccctgccctcccggctccgaatcactatcgccatcaacaacacactcacccccaccaccctgccaccagggaccatccaatacctgactgacacctccaagtatcccaagggttactttgtccagaacacatattttgactttttcaactacgctggactgcagcggtctgtacttctgtacacgacacccaccacctacatcgatgacatcaccgtcaccaccagcgtggagcaagacagtgggctggtgaattaccagatctctgtcaagggcagtaacctgttcaagttggaagtgcgtcttttggatgcagaaaacaaagtcgtggcgaatgggactgggacccagggccaacttaaggtgccaggtgtcagcctctggtggccgtacctgatgcacgaacgccctgcctatctgtattcattggaggtgcagctgactgcacagacgtcactggggcctgtgtctgacttctacacactccctgtggggatccgcactgtggctgtcaccaagagccagttcctcatcaatgggaaacctttctatttccacggtgtcaacaagcatgaggatgcggacatccgagggaagggcttcgactggccgctgctggtgaaggacttcaacctgcttcgctggcttggtgccaacgctttccgtaccagccactacccctatgcagaggaagtgatgcagatgtgtgaccgctatgggattgtggtcatcgatgagtgtcccggcgtgggcctggcgctgccgcagttcttcaacaacgtttctctgcatcaccacatgcaggtgatggaagaagtggtgcgtagggacaagaaccaccccgcggtcgtgatgtggtctgtggccaacgagcctgcgtcccacctagaatctgctggctactacttgaagatggtgatcgctcacaccaaatccttggacccctcccggcctgtgacctttgtgagcaactctaactatgcagcagacaagggggctccgtatgtggatgtgatctgtttgaacagctactactcttggtatcacgactacgggcacctggagttgattcagctgcagctggccacccagtttgagaactggtataagaagtatcagaagcccattattcagagcgagtatggagcagaaacgattgcagggtttcaccaggatccacctctgatgttcactgaagagtaccagaaaagtctgctagagcagtaccatctgggtctggatcaaaaacgcagaaaatatgtggttggagagctcatttggaattttgccgatttcatgactgaacagtcaccgacgagagtgctggggaataaaaaggggatcttcactcggcagagacaaccaaaaagtgcagcgttccttttgcgagagagatactggaagattgccaatgaaaccaggtatccccactcagtagccaagtcacaatgtttggaaaacagcccgtttact

SEQ ID NO: 13 - protein, 663 aa, Homo sapiens
MRGPSGALWLLLALRTVLGSEEEEEEEEAEAETGLQGGMLYPQESPSRECKELDGLWSFRADFSDNRRRGFEEQWYRRPLWESGPTVDMPVPSSFNDISQDWRLRHFVGWVWYEREVILPERWTQDLRTRVVLRIGSAHSYAIVWVNGVDTLEHEGGYLPFEADISNLVQVGPLPSRLRITIAINNTLTPTTLPPGTIQYLTDTSKYPKGYFVQNTYFDFFNYAGLQRSVLLYTTPTTYIDDITVTTSVEQDSGLVNYQISVKGSNLFKLEVRLLDAENKVVANGTGTQGQLKVPGVSLWWPYLMHERPAYLYSLEVQLTAQTSLGPVSDFYTLPVGIRTVAVTKSQFLINGKPFYFHGVNKHEDADIRGKGFDWPLLVKDFNLLRWLGANAFRTSHYPYAEEVMQMCDRYGIVVIDECPGVGLALPQFFNNVSLHHHMQVMEEVVRRDKNHPAVVMWSVANEPASHLESAGYYLKMVIAHTKSLDPSRPVTFVSNSNYAADKGAPYVDVICLNSYYSWYHDYGHLELIQLQLATQFENWYKKYQKPIIQSEYGAETIAGFHQDPPLMFTEEYQKSLLEQYHLGLDQKRRKYVVGELIWNFADFMTEQSPTRVLGNKKGIFTRQRQPKSAAFLLRERYWKIANETRYPHSVAKSQCLENSPFT

SEQ ID NO: 14 - DNA, 1983 bp, Homo sapiens
  CDS (1)..(1983)
atgcggggtccgagcggggctctgtggctgctcctggctctgcgcaccgtgctcggatcagatgatgatgatgatgatgccgaggcagaaaccggtctgcagggcgggatgctgtacccccaggagagcccgtcgcgggagtgcaaggagctggacggcctctggagcttccgcgccgacttctctgacaaccgacgccggggcttcgaggagcagtggtaccggcggccgctgtgggagtcaggccccaccgtggacatgccagttccctccagcttcaatgacatcagccaggactggcgtctgcggcattttgtcggctgggtgtggtacgaacgggaggtgatcctgccggagcgatggacccaggacctgcgcacaagagtggtgctgaggattggcagtgcccattcctatgccatcgtgtgggtgaatggggtcgacacgctagagcatgaggggggctacctccccttcgaggccgacatcagcaacctggtccaggtggggcccctgccctcccggctccgaatcactatcgccatcaacaacacactcacccccaccaccctgccaccagggaccatccaatacctgactgacacctccaagtatcccaagggttactttgtccagaacacatattttgactttttcaactacgctggactgcagcggtctgtacttctgtacacgacacccaccacctacatcgatgacatcaccgtcaccaccagcgtggagcaagacagtgggctggtgaattaccagatctctgtcaagggcagtaacctgttcaagttggaagtgcgtcttttggatgcagaaaacaaagtcgtggcgaatgggactgggacccagggccaacttaaggtgccaggtgtcagcctctggtggccgtacctgatgcacgaacgccctgcctatctgtattcattggaggtgcagctgactgcacagacgtcactggggcctgtgtctgacttctacacactccctgtggggatccgcactgtggctgtcaccaagagccagttcctcatcaatgggaaacctttctatttccacggtgtcaacaagcatgaggatgcggacatccgagggaagggcttcgactggccgctgctggtgaaggacttcaacctgcttcgctggcttggtgccaacgctttccgtaccagccactacccctatgcagaggaagtgatgcagatgtgtgaccgctatgggattgtggtcatcgatgagtgtcccggcgtgggcctggcgctgccgcagttcttcaacaacgtttctctgcatcaccacatgcaggtgatggaagaagtggtgcgtagggacaagaaccaccccgcggtcgtgatgtggtctgtggccaacgagcctgcgtcccacctagaatctgctggctactacttgaagatggtgatcgctcacaccaaatccttggacccctcccggcctgtgacctttgtgagcaactctaactatgcagcagacaagggggctccgtatgtggatgtgatctgtttgaacagctactactcttggtatcacgactacgggcacctggagttgattcagctgcagctggccacccagtttgagaactggtataagaagtatcagaagcccattattcagagcgagtatggagcagaaacgattgcagggtttcaccaggatccacctctgatgttcactgaagagtaccagaaaagtctgctagagcagtaccatctgggtctggatcaaaaacgcagaaaatatgtggttggagagctcatttggaattttgccgatttcatgactgaacagtcaccgacgagagtgctggggaataaaaaggggatcttcactcggcagagacaaccaaaaagtgcagcgttccttttgcgagagagatactggaagattgccaatgaaaccaggtatccccactcagtagccaagtcacaatgtttggaaaacagcccgtttact

SEQ ID NO: 15 - protein, 661 aa, Homo sapiens
MRGPSGALWLLLALRTVLGSDDDDDDAEAETGLQGGMLYPQESPSRECKELDGLWSFRADFSDNRRRGFEEQWYRRPLWESGPTVDMPVPSSFNDISQDWRLRHFVGWVWYEREVILPERWTQDLRTRVVLRIGSAHSYAIVWVNGVDTLEHEGGYLPFEADISNLVQVGPLPSRLRITIAINNTLTPTTLPPGTIQYLTDTSKYPKGYFVQNTYFDFFNYAGLQRSVLLYTTPTTYIDDITVTTSVEQDSGLVNYQISVKGSNLFKLEVRLLDAENKVVANGTGTQGQLKVPGVSLWWPYLMHERPAYLYSLEVQLTAQTSLGPVSDFYTLPVGIRTVAVTKSQFLINGKPFYFHGVNKHEDADIRGKGFDWPLLVKDFNLLRWLGANAFRTSHYPYAEEVMQMCDRYGIVVIDECPGVGLALPQFFNNVSLHHHMQVMEEVVRRDKNHPAVVMWSVANEPASHLESAGYYLKMVIAHTKSLDPSRPVTFVSNSNYAADKGAPYVDVICLNSYYSWYHDYGHLELIQLQLATQFENWYKKYQKPIIQSEYGAETIAGFHQDPPLMFTEEYQKSLLEQYHLGLDQKRRKYVVGELIWNFADFMTEQSPTRVLGNKKGIFTRQRQPKSAAFLLRERYWKIANETRYPHSVAKSQCLENSPFT

SEQ ID NO: 16 - DNA, 1989 bp, Homo sapiens
  CDS (1)..(1989)
atgcggggtccgagcggggctctgtggctgctcctggctctgcgcaccgtgctcggatcagatgatgatgatgatgatgatgatgccgaggcagaaaccggtctgcagggcgggatgctgtacccccaggagagcccgtcgcgggagtgcaaggagctggacggcctctggagcttccgcgccgacttctctgacaaccgacgccggggcttcgaggagcagtggtaccggcggccgctgtgggagtcaggccccaccgtggacatgccagttccctccagcttcaatgacatcagccaggactggcgtctgcggcattttgtcggctgggtgtggtacgaacgggaggtgatcctgccggagcgatggacccaggacctgcgcacaagagtggtgctgaggattggcagtgcccattcctatgccatcgtgtgggtgaatggggtcgacacgctagagcatgaggggggctacctccccttcgaggccgacatcagcaacctggtccaggtggggcccctgccctcccggctccgaatcactatcgccatcaacaacacactcacccccaccaccctgccaccagggaccatccaatacctgactgacacctccaagtatcccaagggttactttgtccagaacacatattttgactttttcaactacgctggactgcagcggtctgtacttctgtacacgacacccaccacctacatcgatgacatcaccgtcaccaccagcgtggagcaagacagtgggctggtgaattaccagatctctgtcaagggcagtaacctgttcaagttggaagtgcgtcttttggatgcagaaaacaaagtcgtggcgaatgggactgggacccagggccaacttaaggtgccaggtgtcagcctctggtggccgtacctgatgcacgaacgccctgcctatctgtattcattggaggtgcagctgactgcacagacgtcactggggcctgtgtctgacttctacacactccctgtggggatccgcactgtggctgtcaccaagagccagttcctcatcaatgggaaacctttctatttccacggtgtcaacaagcatgaggatgcggacatccgagggaagggcttcgactggccgctgctggtgaaggacttcaacctgcttcgctggcttggtgccaacgctttccgtaccagccactacccctatgcagaggaagtgatgcagatgtgtgaccgctatgggattgtggtcatcgatgagtgtcccggcgtgggcctggcgctgccgcagttcttcaacaacgtttctctgcatcaccacatgcaggtgatggaagaagtggtgcgtagggacaagaaccaccccgcggtcgtgatgtggtctgtggccaacgagcctgcgtcccacctagaatctgctggctactacttgaagatggtgatcgctcacaccaaatccttggacccctcccggcctgtgacctttgtgagcaactctaactatgcagcagacaagggggctccgtatgtggatgtgatctgtttgaacagctactactcttggtatcacgactacgggcacctggagttgattcagctgcagctggccacccagtttgagaactggtataagaagtatcagaagcccattattcagagcgagtatggagcagaaacgattgcagggtttcaccaggatccacctctgatgttcactgaagagtaccagaaaagtctgctagagcagtaccatctgggtctggatcaaaaacgcagaaaatatgtggttggagagctcatttggaattttgccgatttcatgactgaacagtcaccgacgagagtgctggggaataaaaaggggatcttcactcggcagagacaaccaaaaagtgcagcgttccttttgcgagagagatactggaagattgccaatgaaaccaggtatccccactcagtagccaagtcacaatgtttggaaaacagcccgtttact

SEQ ID NO: 17 - protein, 663 aa, Homo sapiens
MRGPSGALWLLLALRTVLGSDDDDDDDDAEAETGLQGGMLYPQESPSRECKELDGLWSFRADFSDNRRRGFEEQWYRRPLWESGPTVDMPVPSSFNDISQDWRLRHFVGWVWYEREVILPERWTQDLRTRVVLRIGSAHSYAIVWVNGVDTLEHEGGYLPFEADISNLVQVGPLPSRLRITIAINNTLTPTTLPPGTIQYLTDTSKYPKGYFVQNTYFDFFNYAGLQRSVLLYTTPTTYIDDITVTTSVEQDSGLVNYQISVKGSNLFKLEVRLLDAENKVVANGTGTQGQLKVPGVSLWWPYLMHERPAYLYSLEVQLTAQTSLGPVSDFYTLPVGIRTVAVTKSQFLINGKPFYFHGVNKHEDADIRGKGFDWPLLVKDFNLLRWLGANAFRTSHYPYAEEVMQMCDRYGIVVIDECPGVGLALPQFFNNVSLHHHMQVMEEVVRRDKNHPAVVMWSVANEPASHLESAGYYLKMVIAHTKSLDPSRPVTFVSNSNYAADKGAPYVDVICLNSYYSWYHDYGHLELIQLQLATQFENWYKKYQKPIIQSEYGAETIAGFHQDPPLMFTEEYQKSLLEQYHLGLDQKRRKYVVGELIWNFADFMTEQSPTRVLGNKKGIFTRQRQPKSAAFLLRERYWKIANETRYPHSVAKSQCLENSPFT

SEQ ID NO: 18 - DNA, 52 bp, Artificial
  Forward primer.  Nt 1-5 random synthetic sequence. Nt 6-52 part of sequence encoding p97 signal.
gtaccgaattcaccgccatgcggggtccgagcggggctctgtggctgctcct

SEQ ID NO: 19 - DNA, 51 bp, Artificial
  Reverse primer.  Nt 1-6 random synthetic sequence. Nt 7-52 part of sequence encoding p97 signal.
gtaccgggatccgagcacggtgcgcagagccaggagcagccacagagcccc